(19) United States
(12) Patent Application Publication
(10) Pub. No.: US 2020/0375646 A1
(43) Pub. Date: Dec. 3, 2020

(54) BONE TAMP AND CORRESPONDING METHOD

(71) Applicant: Arthrex, Inc., Naples, FL (US)

(72) Inventors: Paul Fein, Maynard, MA (US); Zachary Day, Naples, FL (US); Alexander Delmonaco, Billerica, MA (US); Chris Powell, Naples, FL (US); Michael Coughlin, Boise, ID (US)

(21) Appl. No.: 16/428,158

(22) Filed: May 31, 2019

(51) Int. Cl. A61B 17/92; A61B 17/88

(52) U.S. Cl. CPC A61B 17/92; A61B 2017/564; A61B 17/8897; A61B 17/885

(57) ABSTRACT

This disclosure relates to a bone tamp and a corresponding method including the bone tamp sliding over a guide wire projecting from a digit of a foot and protruding from an interphalangeal implant.

### BACKGROUND

This disclosure relates to a bone tamp and a corresponding method.

Orthopedic procedures are often performed to fix deformities of interphalangeal joints of the foot. In such procedures, an implant is delivered into the interphalangeal joint to hold adjacent phalanges relative to one another.

### SUMMARY

This disclosure relates to a bone tamp and a corresponding method. In an example method, a bone tamp slides over a guide wire projecting from a digit of a foot and protruding from an interphalangeal implant. A bone tamp includes a distal end portion defined by a surface having a concave contour. In an embodiment, the concave contour is sized and shaped to correspond to the size and shape of an end of a digit of a foot such that the bone tamp is particularly suited to apply force to a digit of a foot, and in particular to close a gap of an interphalangeal joint.

A method of treatment according to an exemplary aspect of the present disclosure includes, inter alia, sliding a bone tamp over a guide wire projecting from a digit of a foot and protruding from an interphalangeal implant. The bone tamp includes a distal end portion defined by a surface having a concave contour. The method further includes applying a force to the distal end of the digit using the bone tamp.

A bone tamp according to an exemplary aspect of the present disclosure includes, inter alia, a distal end portion and a bore longitudinally extending through the distal end portion. Further, the distal end portion is defined by a surface having a concave contour.

### BRIEF DESCRIPTION OF THE DRAWINGS

FIG. 1 is a perspective view of an example surgical device.

FIG. 2 is a cross-sectional view of a portion of the surgical device.

FIG. 3 illustrates a digit with guide wire projecting therefrom and an example surgical device. FIG. 3 is representative of a condition in which a user is preparing to slide the surgical device over the guide wire.

FIG. 4 illustrates the digit with guide wire projecting therefrom and the surgical device arranged relative to the guide wire. FIG. 4 is representative of a condition in which the surgical device has slid along the guide wire and contacts the digit, and in particular has closed a gap between adjacent phalanges.

### DETAILED DESCRIPTION

This disclosure relates to a bone tamp and a corresponding method. In an example method, the bone tamp is slid over guide wire projecting from a digit of a foot and protruding from an interphalangeal implant. The bone tamp includes a distal end portion defined by a surface having a concave contour. In an embodiment, the concave contour is sized and shaped to correspond to the size and shape of an end of a digit of a foot such that the bone tamp is particularly suited to apply force to a digit of a foot, and in particular to close a gap of an interphalangeal joint.

A method of treatment according to an exemplary aspect of the present disclosure includes, inter alia, sliding a bone tamp over a guide wire projecting from a digit of a foot and protruding from an interphalangeal implant. The bone tamp includes a distal end portion defined by a surface having a concave contour. The method further includes applying a force to the distal end of the digit using the bone tamp.

In a further embodiment, the bone tamp includes a bore receiving the guide wire during the sliding step.

In a further embodiment, the guide wire is K-wire.

In a further embodiment, the step of applying the force includes striking a proximal end of the bone tamp with a tool.

In a further embodiment, the tool is one of a hammer and a mallet.

In a further embodiment, the step of applying the force includes pushing bone tamp.

In a further embodiment, the bone tamp includes a reduced outer diameter section adjacent the distal end portion, and pushing the bone tamp includes grasping the reduced outer diameter section.

In a further embodiment, the surface is sized and shaped to correspond to the size and shape of the distal end of the digit.

In a further embodiment, the surface is symmetrical about an axis of the bone tamp.

In a further embodiment, the implant is configured to fuse the interphalangeal joint.

A bone tamp according to an exemplary aspect of the present disclosure includes, inter alia, a distal end portion and a bore longitudinally extending through the distal end portion. Further, the distal end portion is defined by a surface having a concave contour.

In a further embodiment, the surface is symmetrical about an axis of the bone tamp.

In a further embodiment, an outer perimeter of the surface defines a distal-most end of the bone tamp.

In a further embodiment, the surface is defined by a constant radius.

In a further embodiment, the surface is substantially smooth.

In a further embodiment, a plurality of grooves are defined by an outer diameter of the bone tamp.

In a further embodiment, the bone tamp includes a reduced outer diameter section adjacent the distal end portion.

In a further embodiment, the bone tamp includes a proximal end configured to be hit by a tool.

In a further embodiment, the bone tamp is integrally formed as a single unitary structure.

In a further embodiment, the bone tamp is made of one of polyetherimide, polyphenylsulfone, and stainless steel.

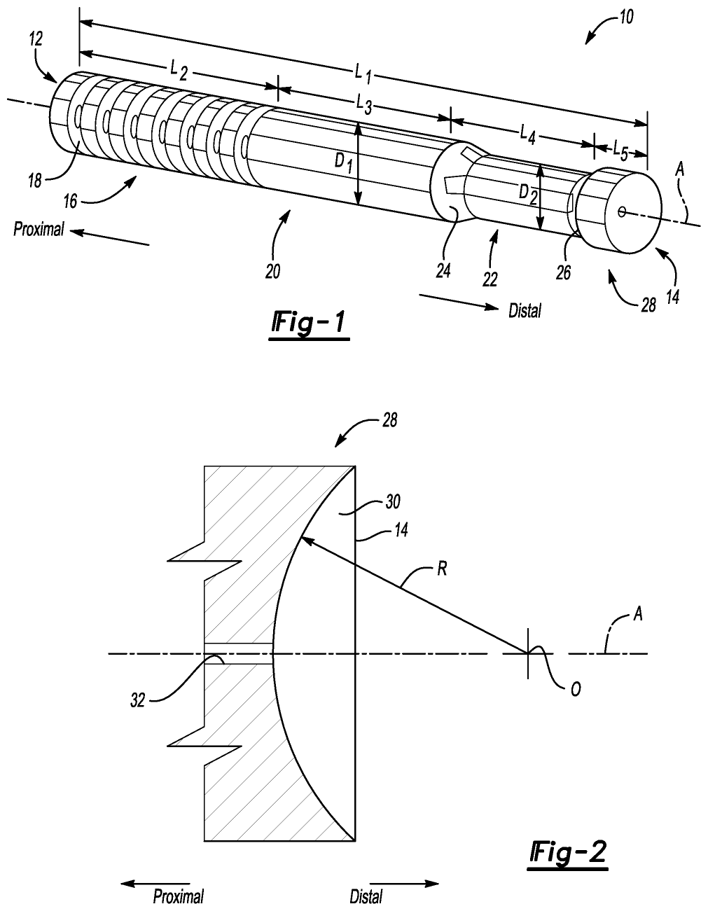

FIG. 1 illustrates an example surgical device 10 according to the present disclosure. In this example, the surgical device 10 is a bone tamp and, in particular, is a toe tamp. The surgical device 10 extends along a central axis A from a proximal end 12 to a distal end 14. The “proximal” and “distal” directions are labeled in FIG. 1 for ease of reference.

The surgical device 10 is substantially cylindrical in this example. In particular, the surgical device 10 exhibits a length L₁ between the proximal end 12 and the distal end 14, and has an outer diameter D₁ along a majority of the length L₁. While the surgical device 10 is substantially cylindrical in this example, this disclosure is not limited to cylindrically-shaped surgical devices.

In one aspect of this disclosure, the surgical device 10 is integrally formed as a single, unitary structure. In other words, the surgical device 10 is a one-piece structure without any seams. The surgical device 10 may be formed from a high strength polymer material such as a polyetherimide like Ultem® or a polyphenylsulfone like Radel®. Alternatively the surgical device 10 may be made of stainless steel.

Beginning at the proximal end 12, the surgical device 10 includes a handle section 16 extending from the proximal end 12 and extending over a length L₂. In the handle section 16, an outer diameter of the surgical device 10 defines a plurality of grooves 18. The grooves 18 extend circumferentially around the entire surgical device 10, in this example. The grooves 18 increase the ease of gripping the surgical device 10. While grooves 18 are shown and described, the surgical device 10 may alternatively or additionally include other features configured to increase the ease of gripping the surgical device 10.

Distal of the handle section 16, the surgical device 10 includes an elongate section 20 extending along a length L₃. The surgical device 10 exhibits a substantially smooth outer contour, without any grooves such as the grooves 20, along the length L₃ in this example. Distal of the length L₃, the surgical device 10 includes a reduced outer diameter section 22 along a length L₄. The reduced outer diameter section 22, in this example, includes a first tapered section 24 in which the outer diameter of the surgical device 10 gradually reduces down from the diameter D₁ to a lesser diameter D₂. The reduced outer diameter section 22 includes a second tapered section 26 adjacent a distal end portion 28 of the surgical device 10, where the outer diameter of the surgical device 10 gradually increases from the diameter D₂ to the diameter D₁. The reduced outer diameter section 22 permits a user (i.e., surgeon) to readily grasp the surgical device along the length L₄ which may be preferable for some users when performing certain procedural steps.

The distal end portion 28 extends from the reduced outer diameter section 22 to the distal end 14 of the surgical device 10 along a length L₅. In this example, the distal end portion 28 is configured to interface with a digit of the human body, and in particular a digit of a foot. More specifically, the distal end portion 28 is configured to directly contact an outer surface of the digit such that the surgical device 10 may transfer force to the digit.

FIG. 2 is a cross-sectional view of the distal end portion 28. As shown in FIG. 2, the distal end portion 28 is defined by a surface 30 having a concave contour. Specifically, the surface 30 is concave when viewed from a location distal to the surgical device 10. The surface 30 is sized and shaped to correspond to the size and shape of a distal end of a digit of a foot. The surface 30 defines a substantially semi-spherical shape, is substantially smooth, and is symmetrical about the axis A in this example. In particular, the surface 30 is defined by a radius R having an origin O on the axis A and spaced-apart distally from the distal end 14 of the surgical device 10. The outer perimeter of the surface 30 defines the distal-most end of the surgical device 10, in this example. While a particular contour of the surface 30 has been shown and described, the distal end portion 28 may exhibit a different contour.

The surgical device 10 further includes a bore 32 extending through the entirety of the surgical device 10 from the proximal end 12 to the surface 30. The bore 32 is coaxial with the axis A and is sized and shaped to correspond with the size and shape of a surgical guide wire.

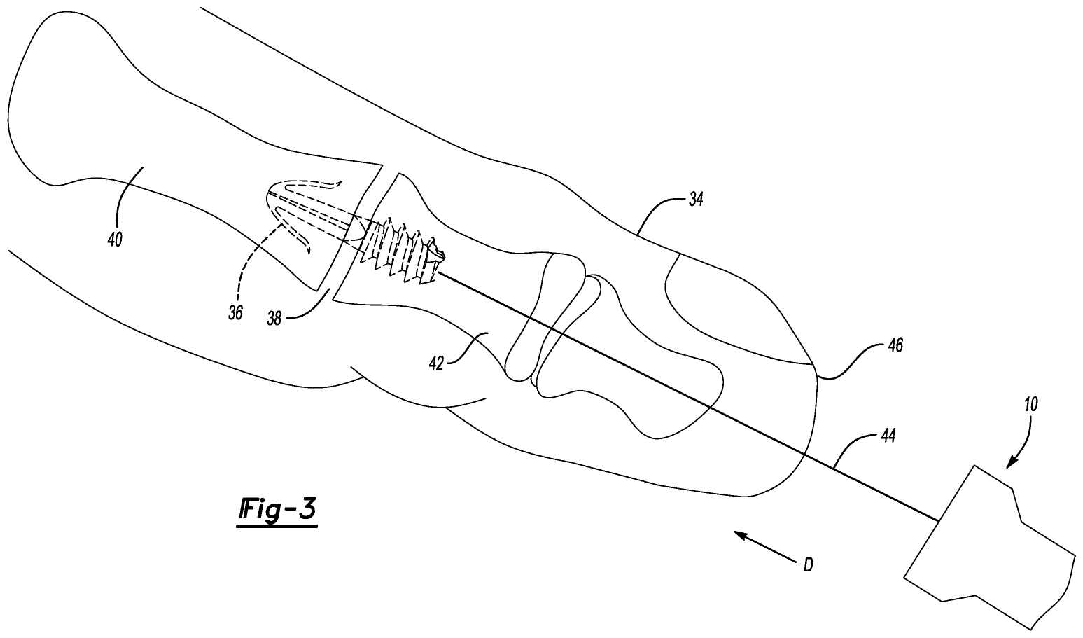

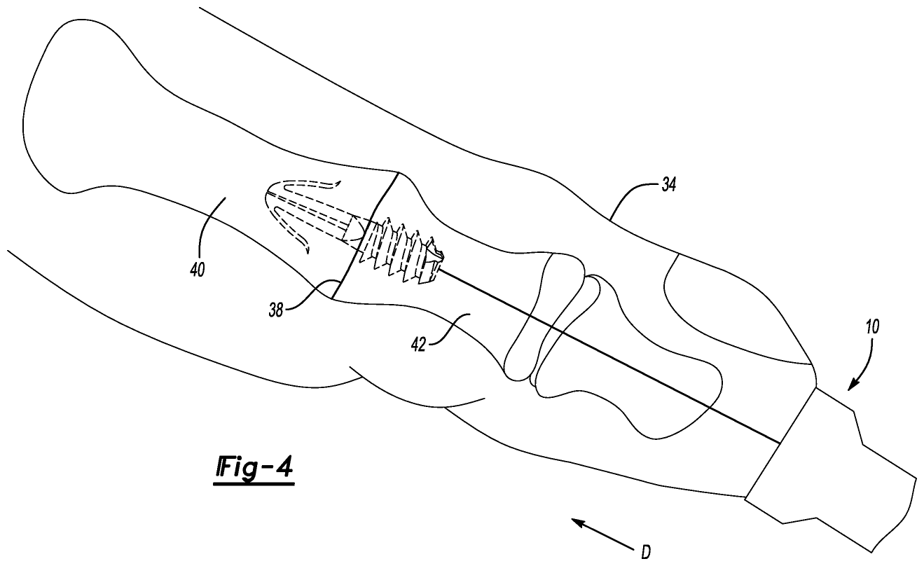

A method of treatment in which the surgical device 10 may be used will now be described with reference to FIGS. 3 and 4. FIG. 3 illustrates a digit 34 (i.e., a toe) of a foot. In FIG. 3, an interphalangeal implant 36 has been delivered into an interphalangeal joint 38 between adjacent phalanges 40, 42. The first phalange 40 may be a proximal phalange and the second phalange 42 may be a middle phalange. The implant 36 may be a hammertoe implant. This disclosure is not limited to any particular type of implant, however.

In FIG. 3, the phalanges 40, 42 are spaced-apart from one another. In order to closed the gap between the phalanges 40, 42, the surgical device 10 is used to apply a force to the digit 34 in direction D.

In this example, a guide wire 44, such as K-wire, projects from the digit 34, and in particular protrudes from the interphalangeal implant 36. The guide wire may be 1.1 mm guide wire in one example. The guide wire 44 may project from the digit 34 by about 0.5 inches.

A user slides the surgical device 10 over the guide wire 44. In particular, the user aligns the surgical device 10 relative to the guide wire 44 such that the guide wire 44 is received in the bore 32. With the guide wire 44 in the bore 32, the user pushes the surgical device 10 in the direction D such that the distal end portion 28, and in particular the surface 30, directly contacts a distal end 46 of the digit 34, as represented in FIG. 4.

In this position, a user may apply force to the digit 34 by pushing the surgical device 10 in the direction D to close the gap between phalanges 40, 42. Again, the user may find it preferable to grasp the reduced outer diameter section 22 when pushing the surgical device 10. Alternatively or in addition, the user may apply force to the digit 34 by striking the proximal end 12 of the surgical device 10 with a tool, such as a hammer or mallet. When striking the surgical device 10 with a tool, the user may grasp the handle section 16 with the opposite hand as the hand that is grasping the tool.

Regardless, as represented in FIG. 4, applying force to the distal end 46 of the digit 34 in the direction D closes the gap between the phalanges 40, 42. With the gap closed, the surgical device 10 is retracted off the guide wire 44, and the guide wire 44 may be removed from the digit 34, allowing the interphalangeal joint 38 to heal. In particular, in this example, the implant 36 is configured to fuse the interphalangeal joint 38. This disclosure is not limited to a particular procedure, however. Following use in a particular procedure, the surgical device 10 may be sanitized and used again in another procedure. To this end, the polymer materials mentioned above are resistant to steam sanitization processes, for example.

It should be understood that terms such as “distal” and “proximal” used above consistent with the way those terms are used in the art. Further, these terms have been used herein for purposes of explanation, and should not be considered otherwise limiting. Terms such as “generally,” “substantially,” and “about” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples.

One of ordinary skill in this art would understand that the above-described embodiments are exemplary and non-limiting. That is, modifications of this disclosure would come within the scope of the claims. Accordingly, the following claims should be studied to determine their true scope and content.

1. A method of treatment, comprising:
sliding a bone tamp over a guide wire projecting from a digit of a foot and protruding from an interphalangeal implant, the bone tamp including a distal end portion defined by a surface having a concave contour; and
applying a force to the distal end of the digit using the bone tamp.

2. The method as recited in claim 1, wherein the bone tamp includes a bore receiving the guide wire during the sliding step.

3. The method as recited in claim 2, wherein the guide wire is K-wire.

4. The method as recited in claim 1, wherein the step of applying the force includes striking a proximal end of the bone tamp with a tool.

5. The method as recited in claim 4, wherein the tool is one of a hammer and a mallet.

6. The method as recited in claim 1, wherein the step of applying the force includes pushing bone tamp.

7. The method as recited in claim 6, wherein the bone tamp includes a reduced outer diameter section adjacent the distal end portion, and wherein pushing the bone tamp includes grasping the reduced outer diameter section.

8. The method as recited in claim 1, wherein the surface is sized and shaped to correspond to the size and shape of the distal end of the digit.

9. The method as recited in claim 8, wherein the surface is symmetrical about an axis of the bone tamp.

10. The method as recited in claim 1, wherein the implant is configured to fuse the interphalangeal joint.

11. A bone tamp, comprising:
a distal end portion; and
a bore longitudinally extending through the distal end portion, wherein the distal end portion is defined by a surface having a concave contour.

12. The bone tamp as recited in claim 11, wherein the surface is symmetrical about an axis of the bone tamp.

13. The bone tamp as recited in claim 12, wherein an outer perimeter of the surface defines a distal-most end of the bone tamp.

14. The bone tamp as recited in claim 12, wherein the surface is defined by a constant radius.

15. The bone tamp as recited in claim 12, wherein the surface is substantially smooth.

16. The bone tamp as recited in claim 11, wherein a plurality of grooves are defined by an outer diameter of the bone tamp.

17. The bone tamp as recited in claim 11, wherein the bone tamp includes a reduced outer diameter section adjacent the distal end portion.

18. The bone tamp as recited in claim 11, wherein the bone tamp includes a proximal end configured to be hit by a tool.

19. The bone tamp as recited in claim 11, wherein the bone tamp is integrally formed as a single unitary structure.

20. The bone tamp as recited in claim 19, wherein the bone tamp is made of one of polyetherimide, polyphenylsulfone, and stainless steel.
